(19) United States
(12) Patent Application Publication
(10) Pub. No.: US 2003/0225397 A1
(43) Pub. Date: Dec. 4, 2003

(54) Guide wire installation device for catheter

(76) Inventor: Philipp Baechtold, Wayne, NJ (US)

Correspondence Address: KANESAKA AND TAKEUCHI, 1423 Powhatan Street, Alexandria, VA, 22314, US

(21) Appl. No.: 10/160,274

(22) Filed: Jun. 4, 2002

(51) Int. Cl. A61B 17/00

(52) U.S. Cl. 606/1

(57) ABSTRACT

A guide wire installation device for a catheter is formed of a catheter receiving section and a guide wire insertion section connected to the catheter receiving section. The catheter receiving section includes a first dent for receiving a distal end of the catheter, a first hole extending from the first dent, and a first slit extending laterally from the first dent and the first hole. The guide wire insertion section includes a second dent for inserting a guide wire, a second hole extending from the second dent and communicating substantially linearly with the first hole, and a second slit extending laterally from the second dent and the second hole. The guide wire can be easily inserted into the catheter through the installation device.

### BACKGROUND OF THE INVENTION AND RELATED ART STATEMENT

The present invention relates to a guide wire installation device for a catheter in installing the catheter onto a guide wire in a percutaneous transluminal coronary angioplasty (PTCA) procedure.

A catheter, such as balloon catheter, has been widely used for vascularization of a coronary artery or delivering and placing a stent in the coronary artery or other portions. In this case, a catheter is delivered into the artery through a guide wire already placed in the artery to enlarge a stenosis, i.e. constriction in the coronary artery, or deliver and place the stent.

In particular, in delivering the catheter into the blood vessel, at first, a through hole for the guide wire in the catheter must be filled with saline to prevent air from entering into the blood vessel, and then, the guide wire is inserted into the through hole of the catheter. Thereafter, the catheter is inserted into the blood vessel through the guide wire.

In this respect, generally, the guide wire has the diameter of only 0.014 inches, and the catheter tip has an opening for the through hole of 0.015 inches. Therefore, it is difficult to quickly enter the guide wire into the opening of the catheter.

Especially, it is desirable to quickly prepare the catheter for treatment to shorten the operation time. Also, a catheter used for one procedure may be exchanged with another catheter, and in this case, the catheter must be prepared as quickly as possible. However, there is no proper device to help using the catheter in installing the guide wire.

The present invention has been made in view of the above problems, and an object of the invention is to provide a guide wire installation device for a catheter for help inserting the guide wire into a through hole of the catheter.

Another object of the invention is to provide a guide wire installation device as stated above, which can be manufactured easily at a low cost.

Further objects and advantages of the invention will be apparent from the following description of the invention.

### SUMMARY OF THE INVENTION

A guide wire installation device for a catheter of the invention is formed of a catheter receiving section, and a guide wire insertion section connected to the catheter receiving section. The catheter receiving section includes a first dent for receiving a distal end of the catheter, a first hole extending from the first dent, and a first slit extending laterally from the first dent and the first hole. The guide wire insertion section includes a second dent for inserting a guide wire, a second hole extending from the second dent and communicating substantially linearly with the first hole, and a second slit extending laterally from the second dent and the second hole.

Preferably, the catheter receiving section and the guide wire insertion section are integrally formed together to have an elongated shape. The first and second dents are located at the opposite sides of the installation device, and the first and second holes extend substantially linearly.

Also, preferably, the first slit is formed horizontally at one side of the catheter receiving section, and the second slit is formed horizontally at one side of the guide wire insertion section opposite to the first slit relative to the first and second holes. A communication or vertical slit is formed between the catheter receiving section and the guide wire insertion section to communicate and intersect the first and second slits. The communication slit extends substantially perpendicularly to the first and second holes.

When a guide wire is inserted into the through hole of the catheter, the tip of the catheter is inserted into the first dent of the catheter insertion section. Then, the guide wire is inserted into the second dent to pass through the first and second holes and enter the through hole of the catheter. Since the size of the second dent is greater than that of the second hole, the guide wire can be easily inserted into the through hole of the catheter through the second dent and the first and second holes. After the guide wire is inserted into the catheter, the catheter is pulled out from the installation device, and the insertion device is turned horizontally about 90 degrees. Thus, the guide wire passes through the first and second slits, and is located in the communication slit. Then, the installation device is pushed or pulled to remove the guide wire from the communication slit. Thereafter, the catheter is inserted into the blood vessel along the guide wire.

Preferably, the catheter receiving section is formed of a first base and a first cover attached to the first base, and the guide wire insertion section is formed of a second base integrally formed with the first base and a second cover attached to the second base. The communication slit is located between the first and second covers.

Preferably, the catheter receiving section and the guide wire insertion section are formed symmetrically with respect to a center point between the first and second holes. Namely, the catheter receiving section and the guide wire insertion section are the same, and arranged symmetrically. Therefore, one of the sections may be used as the catheter receiving section, and the other of the sections may be used as the guide wire insertion section.

### BRIEF DESCRIPTION OF THE DRAWINGS

FIG. 1 is a perspective view showing a guide wire installation device of the present invention;

FIG. 2 is a horizontal sectional view of the guide wire installation device of the invention;

FIG. 3 is a sectional view taken along line 3-3 in FIG. 2; and

FIG. 4 is a sectional view taken along line 4-4 in FIG. 2.

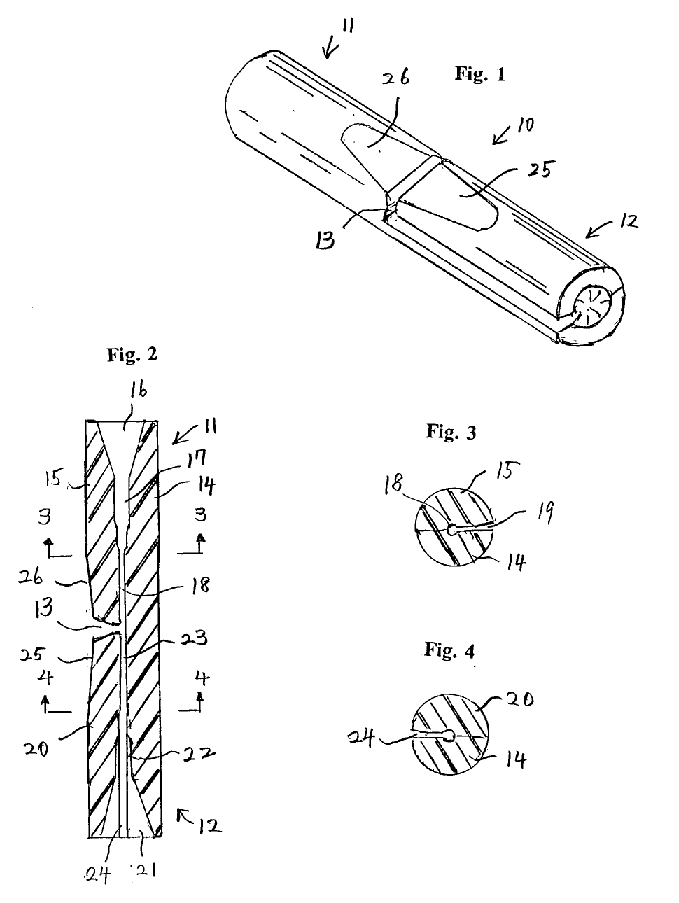

### DETAILED DESCRIPTION OF PREFERRED EMBODIMENTS

Hereinafter, embodiments of the present invention will be explained with reference to the accompanying drawings.

A guide wire installation device 10 of the invention is used in installing a catheter over a guide wire for PTCA procedure, delivering a stent and so on. Namely, the guide wire can be easily inserted into a through hole of the catheter filled with saline to prevent air from entering into a blood vessel when the catheter is delivered into the blood vessel.

The guide wire installation device 10 is formed of a catheter receiving section 11, and a guide wire insertion section 12 integrally formed together with a vertical slit 13 therebetween. In the guide wire installation device 10, the catheter receiving section 11 and the guide wire insertion section 12 are formed symmetrically with respect to a center therebetween.

In particular, the catheter receiving section 11 is formed of a base 14 and a cover 15 disposed over the base 14, and includes a tapered portion or dent 16, a stepped portion 17 and a through hole 18, which are located in the center of the catheter receiving section 11. Also, a slit 19 extends laterally from the tapered portion 16, stepped portion 17 and through hole 18. The vertical thickness of the slit 19 in FIG. 3 is greater than the diameter of the through hole 18. Namely, the diameter of the through hole 18 is slightly greater than that of the guide wire, and the vertical thickness of the slit 19 is substantially the same as or slightly smaller than the diameter of the guide wire. Thus, the guide wire can easily pass through the through hole 18 but not easily enter the slit 19.

The guide wire insertion section 12 is formed of the base 14, and a cover 20 disposed over the base 14 at a side of the cover 15. The guide wire insertion section 12 includes a tapered portion or dent 21, a stepped portion 22 and a through hole 23, which are located in the center of the guide wire insertion section 12. The through hole 23 communicates with the through hole 18 substantially linearly. Also, a slit 24 extends laterally from the tapered portion 21, stepped portion 22 and through hole 23, and is located at a side opposite to the slit 19. The sizes of the through hole 23 and the slit 24 are the same as those of the through hole 18 and the slit 19.

The vertical slit 13 is located between the covers 15, 20, and communicates with the through holes 18, 23. The width of the slit 13 between the covers 15, 20 is substantially the same as or slightly smaller than the diameter of the through hole 18 or 23.

The catheter receiving section 11 and the guide wire insertion section 12 have outer surfaces 25, 26 inclined toward the slit 13 on both outer sides thereof. The inclined surfaces 25, 26 facilitate the handling or gripping of the guide wire installation device 10 with fingers.

In the guide wire installation device 10, the catheter receiving section 11 and the guide wire insertion section 12 are formed symmetrically. Therefore, either one of the sections 11, 12 may be used as the catheter receiving section 11 and the guide wire insertion section 12.

The guide wire installation device 10 is formed of plastic. The covers 15, 20 may be formed integrally with the base 14 at the respective edges of the covers 15, 20, and then, the covers 15, 16 are folded and fixed to the base 14. On the other hand, the base 14 and the covers 15, 20 may be formed separately and bonded together.

The guide wire installation device 10 is used when a catheter, such as a balloon catheter, is delivered into the blood vessel. In this case, the guide wire has already been delivered into the blood vessel. After saline is filled in the through hole of the catheter so that air does not enter the blood vessel, the guide wire installation device is attached to the tip of the catheter. Namely, the tip of the catheter is simply inserted into the dent 16 of the catheter receiving portion 11. At this time, the tip of the catheter is generally located into the stepped portion 17. Then, the rear end of the guide wire already delivered into the blood vessel is inserted into the dent 21 of the guide wire insertion portion 12 and is pushed to enter into the through hole of the catheter through the holes 23, 18. Since the dent 21 of the guide wire insertion section 12 is tapered to open outwardly, the end of the guide wire can be easily inserted into the holes 23, 18 through the dent 21.

After the guide wire is inserted into the catheter, the tip of the catheter with the guide wire therein is slightly pulled to be disengaged from the guide wire installation device 10. Then, the guide wire installation device 10 is horizontally turned 90 degrees with respect to the guide wire. Thus, the guide wire in the through holes 18, 23, the stepped portions 17, 22 and the dents 16, 21 pass through the slits 19, 24 and enters the vertical slit 13. Then, the guide wire installation device 10 is pushed to be separated from the vertical slit 13. Accordingly, the guide wire installation device 10 can be completely detached from the guide wire. Thereafter, the catheter is inserted into the blood vessel along the guide wire as in the regular procedure.

In the present invention, the catheter can be easily attached to or disposed over the guide wire by the guide wire installation device. The used installation device can be easily removed from the catheter and the guide wire.

While the invention has been explained with reference to the specific embodiments of the invention, the explanation is illustrative and the invention is limited only by the appended claims.

What is claimed is:

1. A guide wire installation device for a catheter, comprising:
a catheter receiving section including a first dent for receiving a distal end of the catheter, a first hole extending from the first dent, and a first slit extending laterally from the first dent and the first hole, and
a guide wire insertion section connected to the catheter receiving section and including a second dent for inserting a guide wire, a second hole extending from the second dent and communicating substantially linearly with the first hole, and a second slit extending laterally from the second dent and the second hole.

2. A guide wire installation device according to claim 1, wherein said catheter receiving section and the guide wire insertion section are integrally connected together to have an elongated form such that the first and second dents are located opposite to each other.

3. A guide wire installation device according to claim 2, wherein said first slit is formed at one side of the catheter receiving section, and the second slit is formed at one side of the guide wire insertion section opposite to the first slit relative to the first and second holes.

4. A guide wire installation device according to claim 3, further comprising a communication slit formed between the catheter receiving section and the guide wire insertion section and communicating between the first and second slits.

5. A guide wire installation device according to claim 4, wherein said catheter receiving section is formed of a first base and a first cover attached to the first base, and said guide wire insertion section is formed of a second base integrally formed with the first base and a second cover attached to the second base so that the communication slit is located between the first and second covers.

6. A guide wire installation device according to claim 5, wherein said catheter receiving section and said guide wire insertion section are formed symmetrically with respect to a center point between the first and second holes.

7. A guide wire installation device according to claim 6, wherein each of the first and second dents is tapered inwardly.
